(19) United States
(12) Patent Application Publication
(10) Pub. No.: US 2018/0280046 A1
(43) Pub. Date: Oct. 4, 2018

(54) GUIDEWIRE WITH OPTICS TUBE CONTAINING CORE WIRE

(71) Applicant: Acclarent, Inc., Irvine, CA (US)

(72) Inventors: Don Q. Ngo-Chu, Irvine, CA (US); Wenfeng Lu, Irvine, CA (US); Randy S. Chan, San Jose, CA (US)

(21) Appl. No.: 15/473,761

(22) Filed: Mar. 30, 2017

(51) Int. Cl. A61B 17/24; A61B 90/30; A61B 17/29; A61B 17/221; A61B 34/20; A61M 29/02; A61M 3/02

(52) U.S. Cl. CPC A61B 17/24; A61B 90/30; A61B 17/29; A61B 17/221; A61B 34/20; A61B 2017/246; A61M 3/0279; A61B 2090/306; A61B 2034/2051; A61M 2205/0266; A61M 2205/0238; A61M 29/02

(57) ABSTRACT

An apparatus includes an optics tube extending from a proximal end to a distal end and a distal tip member positioned proximate the distal end of the optics tube. The optics tube includes a cladding material, a core material encased by the cladding material and configured to transmit light from the proximal end of optics tube to the distal end, and a lumen defined by the core material. A core wire is disposed in the lumen. The core wire is configured to prevent longitudinal stretching of the optics tube.

### BACKGROUND

In some instances, it may be desirable to dilate an anatomical passageway in a patient. This may include dilation of ostia of paranasal sinuses (e.g., to treat sinusitis), dilation of the larynx, dilation of the Eustachian tube, dilation of other passageways within the ear, nose, or throat, etc. One method of dilating anatomical passageways includes using a guide wire and catheter to position an inflatable balloon within the anatomical passageway, then inflating the balloon with a fluid (e.g., saline) to dilate the anatomical passageway. For instance, the expandable balloon may be positioned within an ostium at a paranasal sinus and then be inflated, to thereby dilate the ostium by remodeling the bone adjacent to the ostium, without requiring incision of the mucosa or removal of any bone. The dilated ostium may then allow for improved drainage from and ventilation of the affected paranasal sinus. A system that may be used to perform such procedures may be provided in accordance with the teachings of U.S. Pub. No. 2011/0004057, entitled “Systems and Methods for Transnasal Dilation of Passageways in the Ear, Nose or Throat,” published Jan. 6, 2011, the disclosure of which is incorporated by reference herein. An example of such a system is the Relieva® Spin Balloon Sinuplasty™ System by Acclarent, Inc. of Menlo Park, Calif.

A variable direction view endoscope may be used with such a system to provide visualization within the anatomical passageway (e.g., the ear, nose, throat, paranasal sinuses, etc.) to position the balloon at desired locations. A variable direction view endoscope may enable viewing along a variety of transverse viewing angles without having to flex the shaft of the endoscope within the anatomical passageway. Such an endoscope that may be provided in accordance with the teachings of U.S. Pub. No. 2010/0030031, entitled “Swing Prism Endoscope,” published Feb. 4, 2010, the disclosure of which is incorporated by reference herein. An example of such an endoscope is the Acclarent Cyclops™ Multi-Angle Endoscope by Acclarent, Inc. of Menlo Park, Calif.

While a variable direction view endoscope may be used to provide visualization within the anatomical passageway, it may also be desirable to provide additional visual confirmation of the proper positioning of the balloon before inflating the balloon. This may be done using an illuminating guidewire. Such a guidewire may be positioned within the target area and then illuminated, with light projecting from the distal end of the guidewire. This light may illuminate the adjacent tissue (e.g., hypodermis, subdertnis, etc.) and thus be visible to the naked eye from outside the patient through transcutaneous illumination. For instance, when the distal end is positioned in the maxillary sinus, the light may be visible through the patient's cheek. Using such external visualization to confirm the position of the guidewire, the balloon may then be advanced distally along the guidewire into position at the dilation site. Such an illuminating guidewire may be provided in accordance with the teachings of U.S. Pat. No. 9,155,492, entitled “Sinus Illumination Lightwire Device,” issued Oct. 13, 2015, the disclosure of which is incorporated by reference herein. An example of such an illuminating guidewire is the Relieva Luma Sentry™ Sinus Illumination System by Acclarent, Inc, of Menlo Park, Calif.

It may be desirable to provide easily controlled inflation/deflation of a balloon in dilation procedures, including procedures that will be performed only by a single operator. While several systems and methods have been made and used to inflate an inflatable member such as a dilation balloon, it is believed that no one prior to the inventors has made or used the invention described in the appended claims.

### BRIEF DESCRIPTION OF THE DRAWINGS

While the specification concludes with claims which particularly point out and distinctly claim the invention, it is believed the present invention will be better understood from the following description of certain examples taken in conjunction with the accompanying drawings, in which like reference numerals identify the same elements and in which:

FIG. 1 depicts a side elevational view of an exemplary dilation catheter system;

FIG. 2A depicts a side elevational view of an exemplary illuminating guidewire of the dilation catheter system of FIG. 1;

FIG. 2B depicts a side elevational view of an exemplary guide catheter of the dilation catheter system of FIG. 1;

FIG. 2C depicts a side elevational view of an exemplary dilation catheter of the dilation catheter system of FIG. 1;

FIG. 3 depicts a detailed side elevational view of the illuminating guide wire of FIG. 2A;

FIG. 4 depicts a detailed side cross-sectional view of the illuminating guidewire of FIG. 2A;

FIG. 5 depicts a perspective view of an exemplary endoscope suitable for use with the dilation catheter system of FIG. 1;

FIG. 6 depicts a side elevational view of the distal end of the endoscope of FIG. 5, showing an exemplary range of viewing angles;

FIG. 7A depicts a front view of the guide catheter of FIG. 2B positioned adjacent an ostium of the maxillary sinus;

FIG. 7B depicts a front view of the guide catheter of FIG. 2B positioned adjacent an ostium of the maxillary sinus, with the dilation catheter of FIG. 2C and the illuminating guidewire of FIG. 2A positioned in the guide catheter and a distal portion of the guidewire positioned in the maxillary sinus;

FIG. 7C depicts a front view of the guide catheter of FIG. 2B positioned adjacent an ostium of the maxillary sinus, with the illuminating guidewire of FIG. 2A translated further distally relative to the guide catheter and into the maxillary sinus;

FIG. 7D depicts a front view of the guide catheter of FIG. 2B positioned adjacent an ostium of the maxillary sinus, with the dilation catheter of FIG. 2C translated distally relative to the guide catheter along the illuminating guidewire of FIG. 2A so as to position a balloon of the dilation catheter within the ostium;

FIG. 7E depicts a front view of an ostium of the maxillary sinus, with the ostium having been enlarged by inflation of the balloon of FIG. 7D;

FIG. 8 depicts a schematic perspective view of a modified version of the dilation catheter system of FIG. 1 being used in conjunction with an exemplary image guided navigation system;

FIG. 9 depicts a perspective view of a distal portion of an exemplary alternative guidewire suitable for use with the dilation catheter system of FIG. 1;

FIG. 10 depicts a cross-sectional view of the guidewire of FIG. 9, taken along line 10-10 of FIG. 9;

FIG. 11 depicts a perspective view of the distal portion of a modified version of the guidewire of FIG. 9, with a modified distal tip member having an exemplary channel defined therein so as to allow an exemplary forceps device to extend therefrom;

FIG. 12 depicts another perspective view of the modified guidewire of FIG. 9, with an exemplary basket device extending from the channel of the distal tip member;

FIG. 13 depicts a perspective view of the distal portion of another modified version of the guidewire of FIG. 9, with a modified optics tube having an exemplary opening with the basket device of FIG. 12 disposed therein;

FIG. 14 depicts a cross-sectional end view of another exemplary alternative guidewire suitable for use with the dilation catheter system of FIG. 1; and

FIG. 15 depicts a cross-sectional side view of another exemplary alternative guidewire suitable for use with the dilation catheter system of FIG. 1.

The drawings are not intended to be limiting in any way, and it is contemplated that various embodiments of the invention may be carried out in a variety of other ways, including those not necessarily depicted in the drawings. The accompanying drawings incorporated in and forming a part of the specification illustrate several aspects of the present invention, and together with the description serve to explain the principles of the invention; it being understood, however, that this invention is not limited to the precise arrangements shown.

### DETAILED DESCRIPTION

The following description of certain examples of the invention should not be used to limit the scope of the present invention. Other examples, features, aspects, embodiments, and advantages of the invention will become apparent to those skilled in the art from the following description, which is by way of illustration, one of the best modes contemplated for carrying out the invention. As will be realized, the invention is capable of other different and obvious aspects, all without departing from the invention. For example, while various. Accordingly, the drawings and descriptions should be regarded as illustrative in nature and not restrictive.

It will be appreciated that the terms “proximal” and “distal” are used herein with reference to a clinician gripping a handpiece assembly. Thus, an end effector is distal with respect to the more proximal handpiece assembly. It will be further appreciated that, for convenience and clarity, spatial terms such as “top” and “bottom” also are used herein with respect to the clinician gripping the handpiece assembly. However, surgical instruments are used in many orientations and positions, and these terms are not intended to be limiting and absolute.

It is further understood that any one or more of the teachings, expressions, versions, examples, etc. described herein may be combined with any one or more of the other teachings, expressions, versions, examples, etc. that are described herein. The following-described teachings, expressions, versions, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

I. Overview of Exemplary Dilation Catheter System

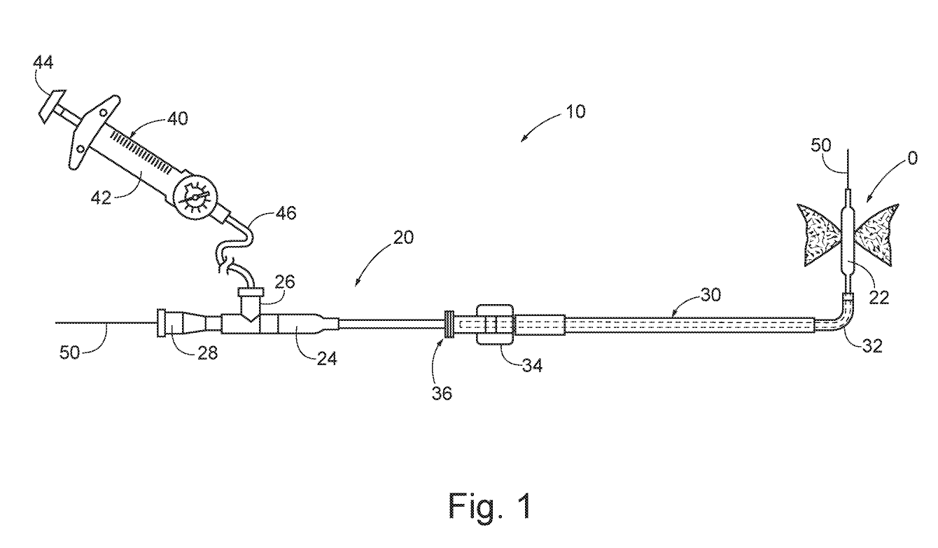

FIG. 1 shows an exemplary dilation catheter system (10) that may be used to dilate the ostium of a paranasal sinus; or to dilate some other anatomical passageway (e.g., within the ear, nose, or throat, etc.). Dilation catheter system (10) of this example comprises a dilation catheter (20), a guide catheter (30), an inflator (40), and a guidewire (50). By way of example only, dilation catheter system (10) may be configured in accordance with at least sonic of the teachings of U.S. Patent Pub. No. 2011/0004057, the disclosure of which is incorporated by reference herein. In some versions, at least part of dilation catheter system (10) is configured similar to the Relieva® Spin Balloon Sinuplasty™ System by Acclarent. Inc. of Menlo Park, Calif.

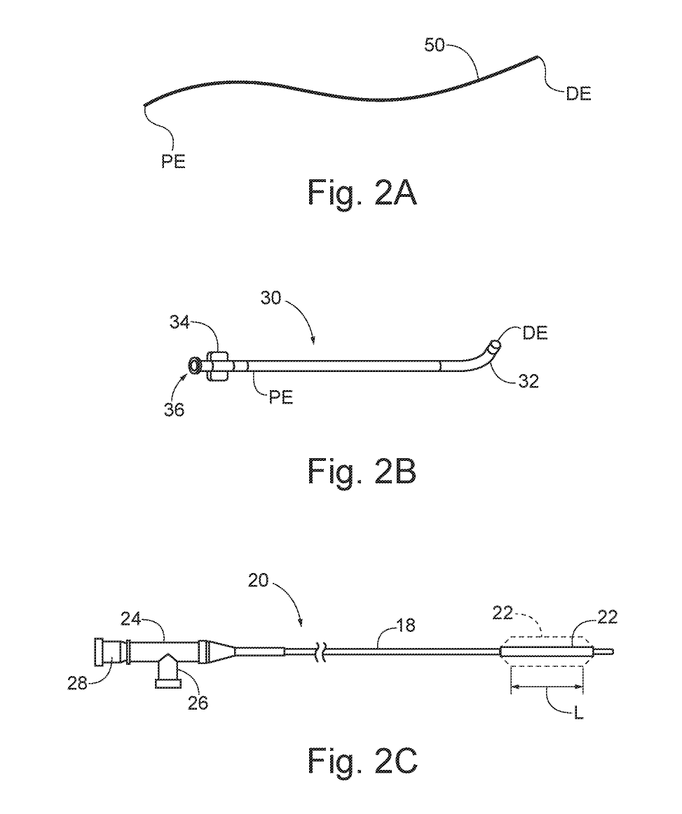

As best seen in FIG. 2C, the distal end (DE) of dilation catheter (20) includes an inflatable dilator (22). The proximal end (PE) of dilation catheter (20) includes a grip (24), which has a lateral port (26) and an open proximal end (28), A hollow-elongate shaft (18) extends distally from grip. Dilation catheter (20) includes a first lumen (not shown) formed within shaft (18) that provides fluid communication between lateral port (26) and the interior of dilator (22). Dilator catheter (20) also includes a second lumen (not shown) formed within shaft (18) that extends from open proximal end (28) to an open distal end that is distal to dilator (22). This second lumen is configured to slidably receive guidewire (50). The first and second lumens of dilator catheter (20) are fluidly isolated from each other. Thus, dilator (22) may be selectively inflated and deflated by communicating fluid along the first lumen via lateral port (26) while guidewire (50) is positioned within the second lumen. In some versions, dilator catheter (20) is configured similar to the Relieva Ultirra™ Sinus Balloon Catheter by Acclarent, Inc. of Menlo Park, Calif. In some other versions, dilator catheter (20) is configured similar to the Relieva Solo Pro™ Sinus Balloon Catheter by Acclarent, Inc. of Menlo Park, Calif. Other suitable forms that dilator catheter (20) may take will be apparent to those of ordinary skill in the art in view of the teachings herein.

As best seen in FIG. 2B, guide catheter (30) of the present example includes a bent distal portion (32) at its distal end (DE) and a grip (34) at its proximal end (PE). Grip (34) has an open proximal end (36). Guide catheter (30) defines a lumen that is configured to slidably receive dilation catheter (20), such that guide catheter (30) may guide dilator (22) out through bent distal end (32). In some versions, guide catheter (30) is configured similar to the Relieva Flex™ Sinus Guide Catheter by Acclarent, Inc., of Menlo Park, Calif. Other suitable forms that guide catheter (30) may take will be apparent to those of ordinary skill in the art in view of the teachings herein.

Referring back to FIG. 1, inflator (40) of the present example comprises a barrel (42) that is configured to hold fluid and a plunger (44) that is configured to reciprocate relative to barrel (42) to selectively discharge fluid from (or draw fluid into) barrel (12). Barrel (42) is fluidly coupled with lateral port (26) via a flexible tube (46). Thus, inflator (40) is operable to add fluid to dilator (22) or withdraw fluid from dilator (22) by translating plunger (44) relative to barrel (42). In the present example, the fluid communicated by inflator (40) comprises saline, though it should be understood that any other suitable fluid may be used. There are various ways in which inflator (40) may be filled with fluid (e.g., saline, etc.). By way of example only, before flexible tube (46) is coupled with lateral port (26), the distal end of flexible tube (16) may be placed in a reservoir containing the fluid. Plunger (44) may then be retracted from a distal position to a proximal position to draw the fluid into barrel (42). Inflator (40) may then be held in an upright position, with the distal end of barrel (42) pointing upwardly, and plunger (44) may then be advanced to an intermediate or slightly distal position to purge any air from barrel (42). The distal end of flexible tube (46) may then be coupled with lateral port (26). In some versions, inflator (40) is constructed and operable in accordance with at least some of the teachings of U.S. Pub. No. 2014/0074141, entitled “Inflator for Dilation of Anatomical Passageway,” published Mar. 13, 2014, the disclosure of which is incorporated by reference herein.

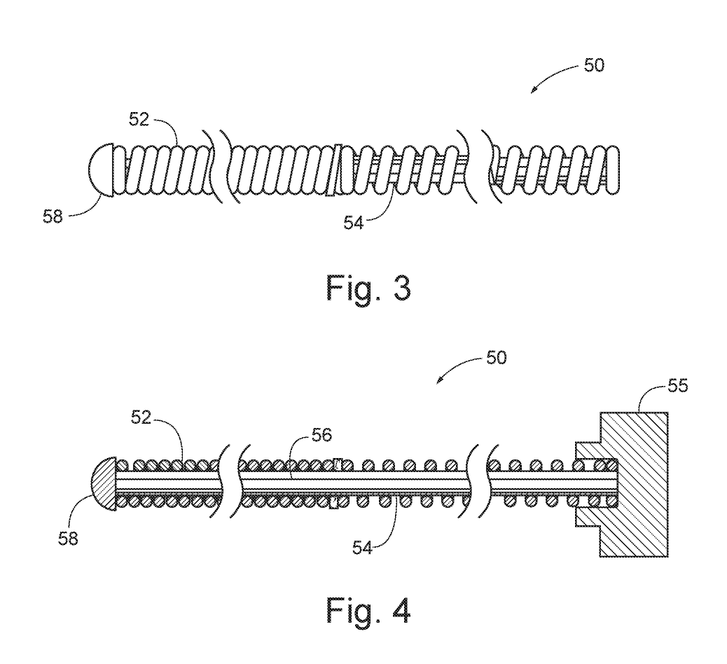

As shown in FIGS. 2A, 3, and 4, guidewire (50) of the present example comprises a coil (52) positioned about a core wire (54). An illumination fiber (56) extends along the interior of core wire (54) and terminates in an atraumatic lens (58). A connector (55) at the proximal end of guidewire (50) enables optical coupling between illumination fiber (56) and a light source (not shown). Illumination fiber (56) may comprise one or more optical fibers. Lens (58) is configured to project light when illumination fiber (56) is illuminated by the light source, such that illumination fiber (56) transmits light from the light source to the lens (58). In some versions, the distal end of guidewire (50) is more flexible than the proximal end of guidewire (50). Guidewire (50) has a length enabling the distal end of guidewire (50) to be positioned distal to dilator (22) while the proximal end of guidewire (50) is positioned proximal to grip (24). Guidewire (50) may include indicia along at least part of its length (e.g., the proximal portion) to provide the operator with visual feedback indicating the depth of insertion of guidewire (50) relative to dilation catheter (20). By way of example only, guidewire (50) may be configured in accordance with at least some of the teachings of U.S. Pat. No. 9,155,492, the disclosure of which is incorporated by reference herein. In some versions, guidewire (50) is configured similar to the Relieva Luma Sentry™ Sinus Illumination System by Acclarent, Inc. of Menlo Park, Calif. Other suitable forms that guidewire (50) may take will be apparent to those of ordinary skill in the art in view of the teachings herein.

II. Overview of Exemplary Endoscope

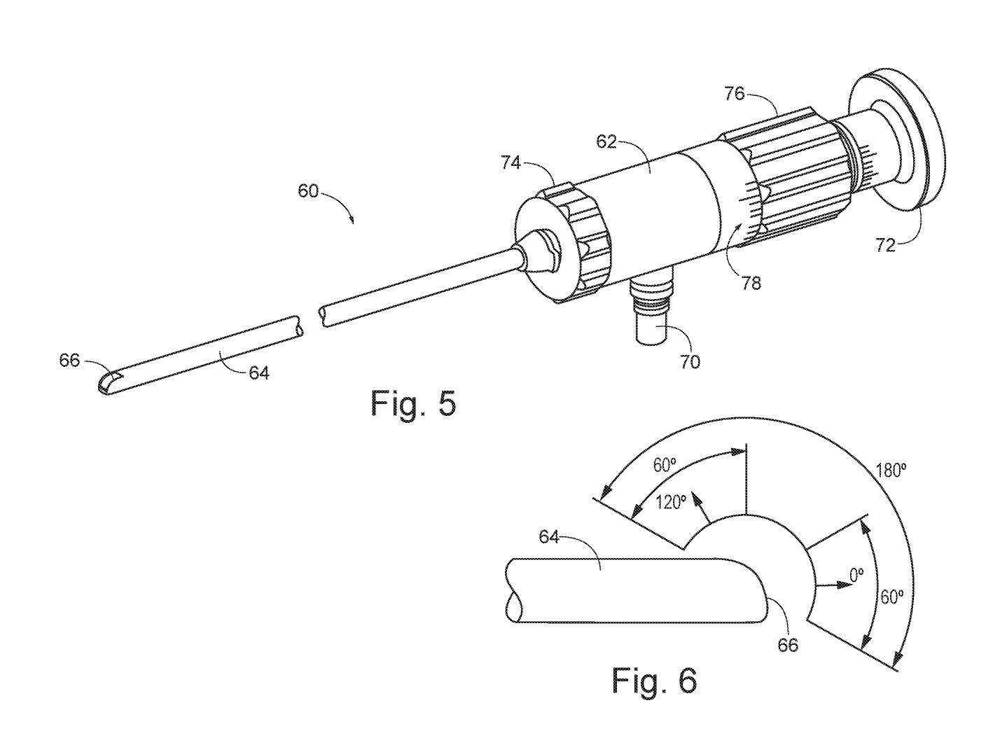

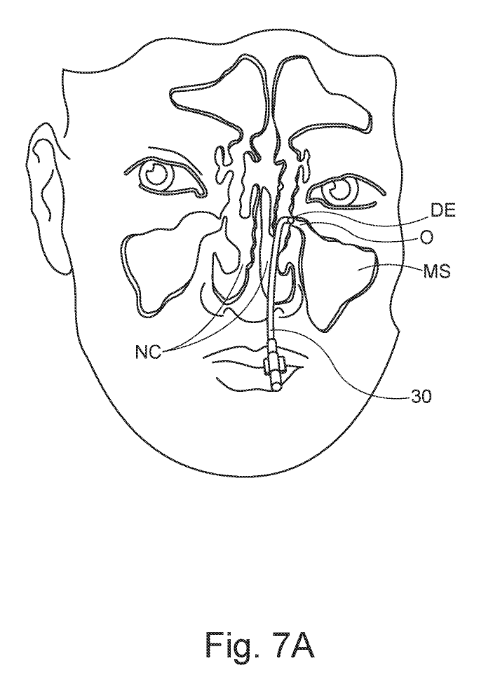

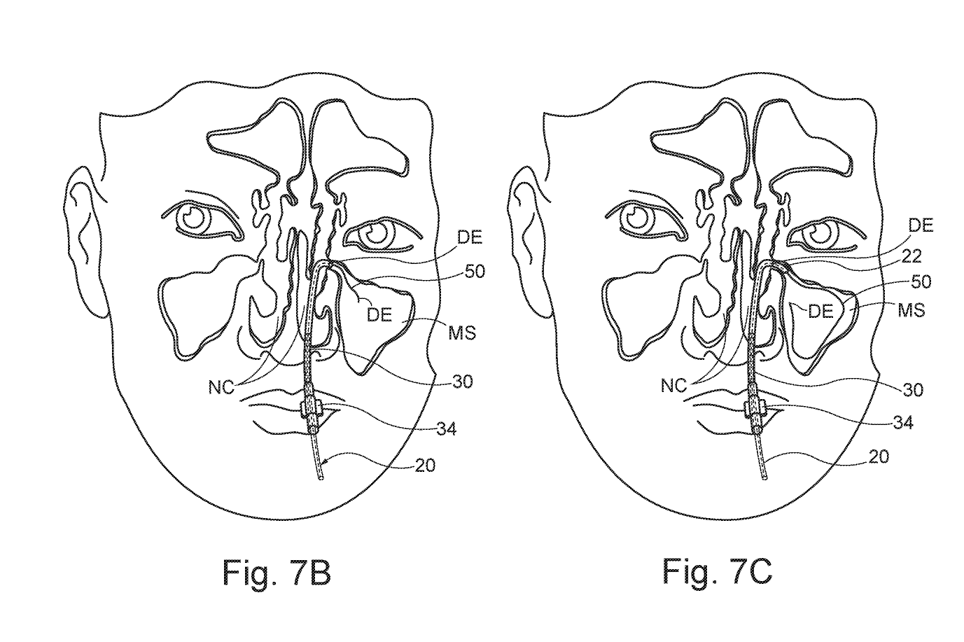

As noted above, an endoscope (60) may be used to provide visualization within an anatomical passageway (e.g., within the nasal cavity, etc.) during a process of using dilation catheter system (10). As shown in FIGS. 4-5, endoscope of the present example comprises a body (62) and a rigid shaft (64) extending distally from body (62). The distal end of shaft (64) includes a curved transparent window (66). A plurality of rod lenses and light transmitting fibers may extend along the length of shaft (64). A lens is positioned at the distal end of the rod lenses and a swing prism is positioned between the lens and window (66). The swing prism is pivotable about an axis that is transverse to the longitudinal axis of shaft (64). The swing prism defines a line of sight that pivots with the swing prism. The line of sight defines a viewing angle relative to the longitudinal axis of shaft (64). This line of sight may pivot from approximately 0 degrees to approximately 120 degrees, from approximately 10 degrees to approximately 90 degrees, or within any other suitable range. The swing prism and window (66) also provide a field of view spanning approximately 60 degrees (with the line of sight centered in the field of view). Thus, the field of view enables a viewing range spanning approximately 180 degrees, approximately 140 degrees, or any other range, based on the pivot range of the swing prism. Of course, all of these values are mere examples.

Body (62) of the present example includes a light post (70), an eyepiece (72), a rotation dial (74), and a pivot dial (76). Light post (70) is in communication with the light transmitting fibers in shaft (64) and is configured to couple with a source of light, to thereby illuminate the site in the patient distal to window (66). Eyepiece (72) is configured to provide visualization of the view captured through window (66) via the optics of endoscope (60). It should be understood that a visualization system (e.g., camera and display screen, etc.) may be coupled with eyepiece (72) to provide visualization of the view captured through window (66) via the optics of endoscope (60). Rotation dial (74) is configured to rotate shaft (64) relative to body (62) about the longitudinal axis of shaft (64). It should be understood that such rotation may be carried out even while the swing prism is pivoted such that the line of sight is non-parallel with the longitudinal axis of shaft (64). Pivot dial (76) is coupled with the swing prism and is thereby operable to pivot the swing prism about the transverse pivot axis. Indicia (78) on body (62) provide visual feedback indicating the viewing angle. Various suitable components and arrangements that may be used to couple rotation dial (74) with the swing prism will be apparent to those of ordinary skill in the art in view of the teachings herein. By way of example only, endoscope (60) may be configured in accordance with at least some of the teachings of U.S. Pub. No. 2010/0030031, the disclosure of which is incorporated by reference herein. In some versions, endoscope (60) is configured similar to the Acclarent Cyclops™ Multi-Angle Endoscope by Acclarent, Inc. of Menlo Park, Calif. Other suitable forms that endoscope (60) may take will be apparent to those of ordinary skill in the art in view of the teachings herein.

III. Exemplary Method for Dilating the Ostium of a Maxillary Sinus

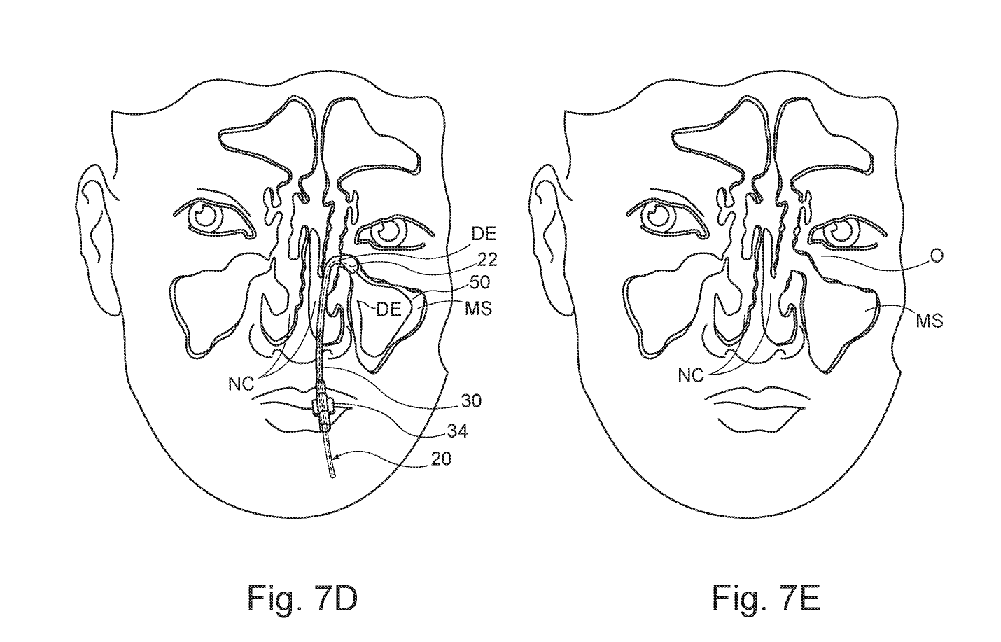

FIGS. 7A-7E show an exemplary method for using dilation catheter system (10) discussed above to dilate a sinus ostium (O) of a maxillary sinus (MS) of a patient. While the present example is being provided in the context of dilating a sinus ostium (O) of a maxillary sinus (MS), it should be understood that dilation catheter system (10) may be used in various other procedures. By way of example only, dilation catheter system (10) and variations thereof may be used to dilate a Eustachian tube, a larynx, a choana, a sphenoid sinus ostium, one or more openings associated with one or more ethmoid sinus air cells, the frontal recess, and/or other passageways associated with paranasal sinuses. Other suitable ways in which dilation catheter system (10) may be used will be apparent to those of ordinary skill in the art in view of the teachings herein.

In the procedure of the present example, guide catheter (30) may be inserted. transnasally and advanced through the nasal cavity (NC) to a position within or near the targeted anatomical passageway to be dilated, the sinus ostium (O), as shown in FIG. 7A. Inflatable dilator (22) and the distal end of guidewire (50) may be positioned within or proximal to bent distal end (32) of guide catheter (30) at this stage. This positioning of guide catheter (30) may be verified endoscopically with an endoscope such as endoscope (60) described above and/or by direct visualization, radiography, and/or by any other suitable method. After guide catheter (30) has been positioned, the operator may advance guidewire (50) distally through guide catheter (30) such that a distal portion of the guidewire (50) passes through the ostium (O) of the maxillary sinus (MS) and into the cavity of the maxillary sinus (MS) as shown in FIGS. 7B and 7C. The operator may illuminate illumination fiber (56) and lens (58), which may provide transcutaneous illumination through the patient's face to enable the operator to visually confirm positioning of the distal end of guidewire (50) in the maxillary sinus (MS) with relative ease.

As shown in FIG. 7C, with guide catheter (30) and guidewire (50) suitably positioned, dilation catheter (20) is advanced along guidewire (50) and through bent distal end (32) of guide catheter (30), with dilator (22) in a non-dilated state until dilator (22) is positioned within the ostium (O) of the maxillary sinus (MS) (or sonic other targeted anatomical passageway). After dilator (22) has been positioned within the ostium (O), dilator (22) may be inflated, thereby dilating the ostium (O), as shown in FIG. 7D. To inflate dilator (22), plunger (44) may be actuated to push saline from barrel (42) of inflator (40) through dilation catheter (20) into dilator (22). The transfer of fluid expands dilator (22) to an expanded state to open or dilate the ostium (O), such as by remodeling the bone, etc., forming ostium (O). By way of example only, dilator (22) may be inflated to a volume sized to achieve about 10 to about 12 atmospheres. Dilator (22) may be held at this volume for a few seconds to sufficiently open the ostium (O) (or other targeted anatomical passageway). Dilator (22) may then be returned to a non-expanded state by reversing plunger (44) of inflator (40) to bring the saline back to inflator (40). Dilator (22) may be repeatedly inflated and deflated in different ostia and/or other targeted anatomical passageways. Thereafter, dilation catheter (20), guidewire (50), and guide catheter (30) may be removed from the patient as shown in FIG. 7E.

In some instances, it may be desirable to irrigate the sinus and paranasal cavity after dilation catheter (20) has been used to dilate the ostium (O). Such irrigation may be performed to flush out blood, etc. that may be present after the dilation procedure. For example, in some cases, guide catheter (30) may be allowed to remain in place after removal of guidewire (50) and dilation catheter (20) and a lavage fluid, other substance, or one or more other devices (e.g., lavage catheters, balloon catheters, cutting balloons, cutters, chompers, rotating cutters, rotating drills, rotating blades, sequential dilators, tapered dilators, punches, dissectors, burs, non-inflating mechanically expandable members, high frequency mechanical vibrators, dilating stents and radiofrequency ablation devices, microwave ablation devices, laser devices, snares, biopsy tools, scopes, and devices that deliver diagnostic or therapeutic agents) may be passed through guide catheter (30) for further treatment of the condition. By way of example only, irrigation may be carried out in accordance with at least some of the teachings of U.S. Pat. No. 7,630,676, entitled “Methods, Devices and Systems for Treatment and/or Diagnosis of Disorders of the Ear, Nose and Throat,” issued Dec. 8, 2009, the disclosure of which is incorporated by reference herein. An example of an irrigation catheter that may be fed through guide catheter (30) to reach the irrigation site after removal of dilation catheter (20) is the Relieva Vortex® Sinus Irrigation Catheter by Acclarent, Inc. of Menlo Park, Calif. Another example of an irrigation catheter that may be fed through guide catheter (30) to reach the irrigation site after removal of dilation catheter (20) is the Relieva Ultirra® Sinus Irrigation Catheter by Acclarent, Inc. of Menlo Park, Calif. Of course, irrigation may be provided in the absence of a dilation procedure; and a dilation procedure may be completed without also including irrigation.

IV. Exemplary Image Guided Navigation System

Image-guided surgery (IGS) is a technique wherein a computer is used to obtain a real-time correlation of the location of an instrument that has been inserted into a patient's body to a set of preoperatively obtained images (e.g., a CT or MRI scan, 3-D map, etc.) so as to superimpose the current location of the instrument on the preoperatively obtained images. In some IGS procedures, a digital tomographic scan (e.g., CT or MRI, 3-D map, etc.) of the operative field is obtained prior to surgery. A specially programmed computer is then used to convert the digital tomographic scan data into a digital map. During surgery, special instruments having sensors (e.g., electromagnetic coils that emit electromagnetic fields and/or are responsive to externally generated electromagnetic fields) mounted thereon are used to perform the procedure while the sensors send data to the computer indicating the current position of each surgical instrument. The computer correlates the data it receives from the instrument-mounted sensors with the digital map that was created from the preoperative tomographic scan. The tomographic scan images are displayed on a video monitor along with an indicator (e.g., cross hairs or an illuminated dot) showing the real time position of each surgical instrument relative to the anatomical structures shown in the scan images. In this manner, the surgeon is able to know the precise position of each sensor-equipped instrument by viewing the video monitor even if the surgeon is unable to directly visualize the instrument itself at its current location within the body.

Examples of electromagnetic IGS systems that may be used in ENT and sinus surgery include the InstaTrak ENT™ systems available from GE Medical Systems, Salt Lake City, Utah. Other examples of electromagnetic image guidance systems that may be modified for use in accordance with the present disclosure include but are not limited to the CARTO® 3 System by Biosense-Webster, Inc., of Diamond Bar, Calif.; systems available from Surgical Navigation Technologies, Inc., of Louisville, Colo.; and systems available from Calypso Medical Technologies, Inc., of Seattle, Wash.

When applied to functional endoscopic sinus surgery (FESS), balloon sinuplasty, and/or other ENT procedures, the use of image guidance systems allows the surgeon to achieve more precise movement and positioning of the surgical instruments than can be achieved by viewing through an endoscope alone. This is so because a typical endoscopic image is a spatially limited, 2 dimensional, line-of-sight view. The use of image guidance systems provides a real time, 3 dimensional view of all of the anatomy surrounding the operative field, not just that which is actually visible in the spatially limited, 2 dimensional, direct line-of-sight endoscopic view. As a result, image guidance systems may be particularly useful during performance of FESS, balloon sinuplasty, and/or other ENT procedures, especially in cases where normal anatomical landmarks are not present or are difficult to visualize endoscopically.

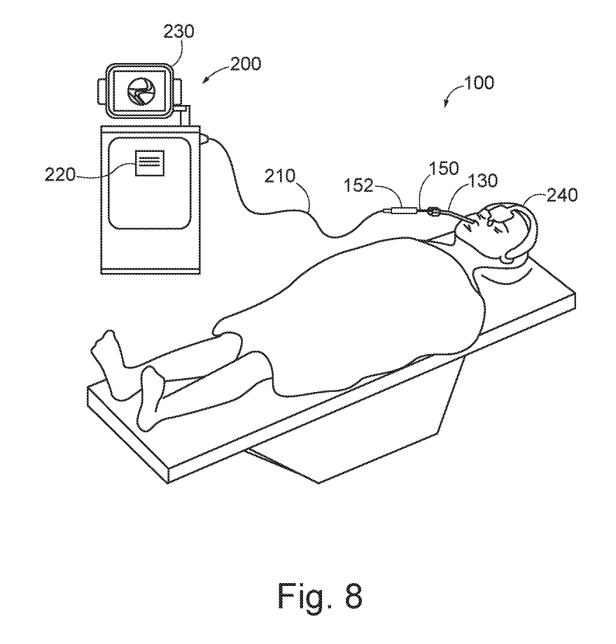

FIG. 8 shows a modified dilation catheter system (100) in combination with an exemplary image guidance system (200). Dilation catheter system (100) comprises a guide catheter (130) with a guidewire (150) slidably disposed therein. Guide catheter (130) may be constructed and operable just like guide catheter (30) described above, such that further details will not be provided here. It should also be understood that, while not shown in FIG. 8, dilation catheter system (100) may also include a dilation catheter that is constructed and operable just like dilation catheter (20) described above. The dilation catheter may be slid along guidewire (150) and through guide catheter (130) as described above.

Guidewire (150) of this example is substantially similar to guidewire (50) described above, except that guidewire (150) of this example is particularly configured to operate in conjunction with navigation system (200). In particular, guidewire (150) includes a connector hub (152) that is configured to couple with a cable (210) of image guidance system (200). The distal end of guidewire (150) includes a coil (not shown) that is in communication with one or more electrical conduits that extend along the length of guidewire (150). When the coil is positioned within an electromagnetic field, movement of the coil within that magnetic field may generate electrical current in the coil, and this electrical current may be communicated along the electrical conduit(s) in guidewire (150) and further along cable (210) via connector hub (152). This phenomenon may enable image guidance system (200) to determine the location of the distal end of guidewire (150) within a three dimensional space as will be described in greater detail below.

While guidewire (150) only has one coil in the present example, it should be understood that guidewire (150) may have two or more coils. Moreover, guidewire (150) may have some other kind of position sensing component that does not necessarily constitute a coil. It should be understood that the distal end of guidewire (150) may be constructed in numerous ways. Several merely illustrative examples of ways in which the distal end of guidewire (150) may be constructed will be described in greater detail below.

Image guidance system (200) of this example further comprises a computer (220), a video display monitor (230), and a field emitting assembly (240). Field emitting assembly (240) is operable to generate an electromagnetic field around the head of the patient. By way of example only, field emitting assembly (240) may comprise a set of coils. Various suitable components that may be used to form and drive field emitting assembly (240) will be apparent to those of ordinary skill in the art in view of the teachings herein. While field emitting assembly (240) is shown as being part of a headset worn by the patient in FIG. 8, it should be understood that field emitting assembly (240) may be positioned at any other suitable location.

Computer (220) includes hardware and software that is configured to drive field emitting assembly (240) and process signals generated by the coil(s) of guidewire (150). In particular, as guidewire (150) is moved within the field generated by field emitting assembly (240), the coil(s) generates position related signals and these signals are communicated to computer (220) via connector hub (152) and cable (210). A processor in computer (220) executes an algorithm to calculate location coordinates of the distal end of guidewire (150) from the position related signals of the coil(s) in guidewire (150). Computer (220) is further operable to provide video in real time via video display monitor (230), showing the position of the distal end of guidewire (150) in relation to a three dimensional model of the anatomy within and adjacent to the patient's nasal cavity.

In some instances, guidewire (150) is used to generate a three dimensional model of the anatomy within and adjacent to the patient's nasal cavity; in addition to being used to provide navigation for dilation catheter system (100) within the patient's nasal cavity. Alternatively, any other suitable device may be used to generate a three dimensional model of the anatomy within and adjacent to the patient's nasal cavity before guidewire (150) is used to provide navigation for dilation catheter system (100) within the patient's nasal cavity. By way of example only, a model of this anatomy may be generated in accordance with at least some of the teachings of U.S. Pub. No. 2016/0310042, entitled “System and Method to Map Structures of Nasal Cavity,” published Oct. 27, 2016, the disclosure of which is incorporated by reference herein. Still other suitable ways in which a three dimensional model of the anatomy within and adjacent to the patient's nasal cavity may be generated will be apparent to those of ordinary skill in the art in view of the teachings herein. It should also be understood that, regardless of how or where the three dimensional model of the anatomy within and adjacent to the patient's nasal cavity is generated, the model may be stored on computer (220). Computer (220) may thus render images of at least a portion of the model via video display monitor (230) and further render real-time video images of the position of guidewire (150) in relation to the model via video display monitor (230).

By way of example only, dilation catheter system (100) and/or image guidance system (200) may be constructed and operable in accordance with at least some of the teachings of U.S. Pat. No. 8,702,626, entitled “Guidewires for Performing Image Guided Procedures,” issued Apr. 22, 2014, the disclosure of which is incorporated by reference herein; U.S. Pat. No. 8,320,711, entitled “Anatomical Modeling from a 3-D Image and a Surface Mapping,” issued Nov. 27, 2012, the disclosure of which is incorporated by reference herein; U.S. Pat. No. 8,190,389, entitled “Adapter for Attaching Electromagnetic Image Guidance Components to a Medical Device,” issued May 29, 2012, the disclosure of which is incorporated by reference herein; U.S. Pat. No. 8,123,722, entitled “Devices, Systems and Methods for Treating Disorders of the Ear, Nose and Throat,” issued Feb. 28, 2012, the disclosure of which is incorporated by reference herein; and U.S. Pat. No. 7,720,521, entitled “Methods and Devices for Performing Procedures within the Ear, Nose, Throat and Paranasal Sinuses,” issued May 18, 2010, the disclosure of which is incorporated by reference herein.

By way of further example only, dilation catheter system (100) and/or image guidance system (200) may be constructed and operable in accordance with at least some of the teachings of U.S. Pat. Pub. No. 2014/0364725, entitled “Systems and Methods for Performing Image Guided Procedures within the Ear, Nose. Throat and Paranasal Sinuses,” published Dec. 11, 2014, the disclosure of which is incorporated by reference herein; U.S. Pat. Pub. No. 2014/0200444, entitled “Guidewires for Performing Image Guided Procedures,” published Jul. 17, 2014, the disclosure of which is incorporated by reference herein; U.S. Pat. No. 9,198,736, entitled “Adapter for Attaching Electromagnetic Image Guidance Components to a Medical Device,” issued Dec. 1, 2015, the disclosure of which is incorporated by reference herein; U.S. Pat. Pub. No. 2011/0060214, entitled “Systems and Methods for Performing Image Guided Procedures within the Ear, Nose, Throat and Paranasal Sinuses,” published Mar. 10, 2011, the disclosure of which is incorporated by reference herein; U.S. Pat. No. 9,167,961, entitled “Methods and Apparatus for Treating Disorders of the Ear Nose and Throat,” issued Oct. 27, 2015, the disclosure of which is incorporated by reference herein; and U.S. Pat. Pub. No. 2007/0208252, entitled “Systems and Methods for Performing Image Guided Procedures within the Ear, Nose, Throat and Paranasal Sinuses,” published Sep. 6, 2007, the disclosure of which is incorporated by reference herein.

It should be understood from the foregoing that the combination of dilation catheter system (100) and image guidance system (200) may be used to perform image guided dilation procedures within the ostia of the various paranasal sinuses, within the frontal recess, within the Eustachian tube, and/or within other passageways associated with the ear, nose, and throat. For instance, the combination of dilation catheter system (100) and image guidance system (200) may be used to perform the dilation of the sinus ostium (O) of the maxillary sinus (MS) as shown in FIGS. 7A-7E and described above. Even in instances where an endoscope such as endoscope (60) is used to provide some degree of visualization within the nasal cavity, the addition of the coil sensor in guidewire (150) and the imaging functionality provided through image guidance system (200) may further enhance the experience of the operator by effectively providing further visualization of anatomical regions that cannot be viewed through endoscope (60). Furthermore, the imaging functionality provided through image guidance system (200) may provide further feedback to the operator indicating the positioning of guidewire (150) within the patient with a degree of precision that could not be obtained using a conventional guidewire (50). Other potential benefits and functionalities that may be obtained through using the combination of dilation catheter system (100) and image guidance system (200) will be apparent to those of ordinary skill in the art in view of the teachings herein.

V. Exemplary Capillary Light Guidewire System

As noted above and shown in FIGS. 2A, 3, and 4, the distal end of guidewire (50) includes a lens (58) that is illuminated by light communicated along illumination fiber (56). Illumination fiber (56) is optically coupled with a light source (not shown) to communicate the light to lens (58). Illumination fiber (56) is contained within coil (52), which is formed by a metallic wire that is wrapped in a wound, helical configuration. The inner diameter of coil (52) is large enough to accommodate other components (e.g., core wire (54)) in addition to accommodating illumination fiber (56). In the present example, illumination fiber (56) has a diameter that is too small to fit additional components within illumination fiber (56). Thus, in order to provide light transmission and accommodate multiple components within a single, small diameter, guidewire (50) requires a combination of coil (52) and illumination fiber (56).

In view of the foregoing, it may be desirable to provide a variation of guidewire (50) that effectively combines the structure of illumination fiber (56) with coil (52) in a single component. In particular, it may be desirable to provide a variation of guidewire (50) where a single component is configured to provide light transmission capabilities (like illumination fiber (56)), in addition to being able to accommodate other components within the diameter of the single component (like coil (52)). Providing both functionalities in a single component may improve production costs and reliability. The following description provides merely illustrative examples of how guidewire (50) may be modified to provide light transmission capabilities (like illumination fiber (56)), in addition to being able to accommodate other components within the diameter of the single component (like coil (52)), in a single component. It should be understood that the exemplary guidewires described below may be readily incorporated into dilation catheter system (10, 100) in place of guidewire (50, 130). It should also be understood that the exemplary guidewires described below may be constructed as small diameter catheters that act as light pipes while defining one or more lumens therethrough.

A. Capillary Light Guidewire with A Single Lumen

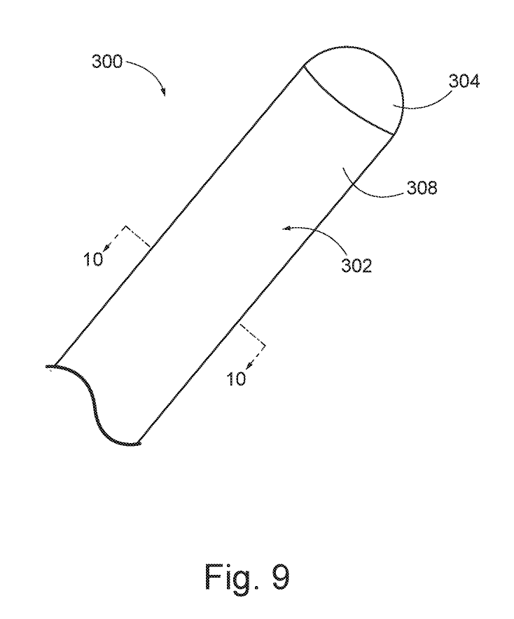

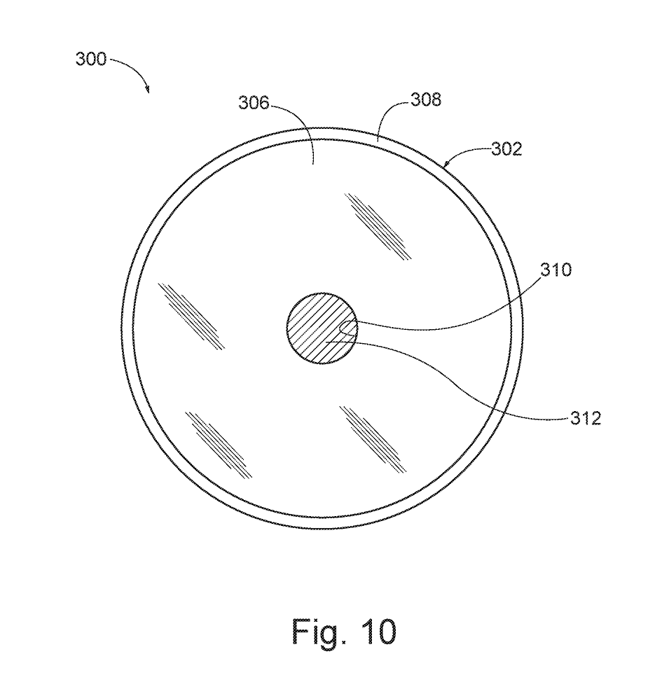

FIGS. 9 and 10 show an exemplary single lumen capillary light guidewire (300) that may be incorporated into dilation catheter system (100) and/or used with image guidance system (200) in place of guidewire (50, 130). Guidewire (300) of this example comprises an optics tube (302) extending from a proximal end to a distal end, with a distal tip member (304) disposed at the distal end thereof. Optics tube (302) includes a core material (306) encased by a cladding material (308).

In the present example, core material (306) defines an internal lumen (310) extending from a proximal end to the distal end of optics tube (302). As shown in FIG. 10, a core wire (312) is slidably disposed within internal lumen (308), such that core wire (312) extends along the entire length of optics tube (302). Optics tube (302) is depicted in FIGS. 9 and 10 as having a circular cross-sectional shape. However, some versions of optics tube (302) are formed in a non-circular cross-sectional shape. While core wire (312) is coaxially aligned with optics tube (302) in the present example, in some other versions, core wire (312) may be laterally offset from the central longitudinal axis of optics tube (302).

Core material (306) is configured to transmit light from light source to the distal end of optics tube (302) in cooperation with cladding material (308). Core material (306) is formed of a generally light transmissive material, while cladding material (308) is formed of a generally light reflective material to contain and prevent leakage of light along the length of optics tube (302). With respect to relative light refraction, in the present example, cladding material (308) has a lower index of refraction than core material (306). Core material (306) may be comprised of silica or plastics such as poly(methyl methacrylate) (also known as PMMA), polystyrene, amorphous fluoropolymer (poly(perfluoro-butenylvinyl ether), or any other suitable optically transmissive material. In some versions, optics tube (302) is free from any metal components.

Core wire (312) is configured to provide additional structural integrity or column strength to optics tube (302) without impacting the light transmissive or optical properties of core material (306). In some versions, the proximal end of core wire (312) is fixedly secured to the proximal end of optics tube (302), while the distal end of core wire (312) is fixedly secured to the distal end of optics tube (302). Core wire (312) is formed of a flexible yet non-extensible m material. Core wire (312) thus prevents or restricts longitudinal stretching of optics tube (302). It should also be understood that core wire (312) may prevent optics tube (302) from kinking. By way of example only, core wire (312) may be configured to prevent optics tube (302) from bending to form an angle of less than approximately 42 degrees.

By way of example only, core wire (312) may be formed from a shape memory alloy such as nickel-titanium alloy, a stainless steel material, a cobalt-chromium alloy, or any combination of these or other materials. In some versions, core wire (312) may have a polytetrafluoroethylene (PTFE) or parylene coating on the outer surface thereof. Various suitable materials and configurations that may be used to form core wire (312) will be apparent to those of ordinary skill in the art in view of the teachings herein.

Distal tip member (304) has an atraumatic dome shape and is secured to the distal end of optics tube (302). By way of example only, distal tip member (304) may be formed of an optically transmissive polymeric material and may be secured to the distal end of optics tube (302) using an interference fit, welding, adhesive, or using any other suitable techniques. As another merely illustrative example, distal tip member (304) may be formed by an optically transmissive adhesive that is applied to the distal end of optics tube (302) and then cured. It should also be understood that distal tip member (304) may be configured and operable like lens (58) described above. Other suitable ways in which distal tip member (304) may be configured and operable will be apparent to those of ordinary skill in the art in view of the teachings herein.

In some versions, the distal end of optics tube (302) is optically coupled with distal tip member (304). The proximal end of optics tube (302) is configured to couple with a light source. Optics tube (302) is configured to provide a path for communication of light from the light source to distal tip member (304), such that distal tip member (304) can emit light generated by the light source. Various suitable ways in which optics tube (302) may be coupled with a light source will be apparent to those of ordinary skill in the art in view of the teachings herein.

In some versions, core wire (312) is movable within internal lumen (310) and may be replaced as desired with other elements such as an optical fiber (not shown) for imaging purposes; or some other kind of instrument that may be used for some other purpose (e.g., biopsy, etc.). Alternatively, one or more additional lumens may be formed in optics tube (302) to accommodate one or more optical fibers and/or other structures, as described in greater detail below or otherwise. In variations of guidewire (300) having an optical fiber positioned within optics tube (302), the optical fiber may be configured to capture an image from the distal end of optics tube (302) and communicate the image to the proximal end of optics tube (302).

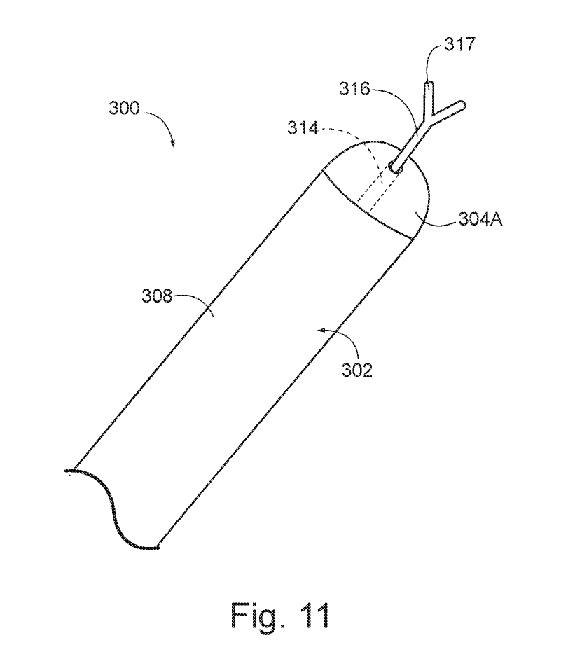

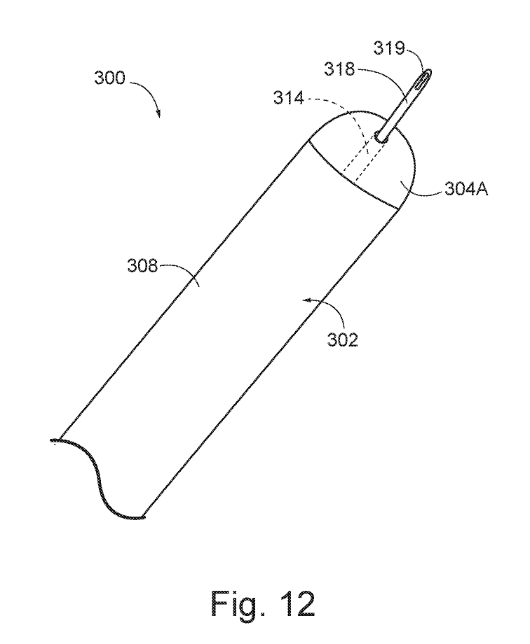

As shown in FIGS. 11 and 12, in some versions of guidewire (300), distal tip member (304) may be replaced with a distal tip member (304A). Distal tip member (304A) defines a channel (314) therein. Channel (314) is in communication with an additional instrument lumen (which is offset from yet parallel with lumen (310)) in optics tube (302) and extends to the exterior or distal end of distal tip member (304A). As shown in FIG. 11, in some versions, the instrument lumen and channel (314) may receive a forceps device (316) therein, with a grasping portion (317) of forceps device (316) being extendable outwardly away from distal tip member (304A) to grasp a portion of the patient's tissue or other structure. The tissue may then be removed from the patient for use in a biopsy or other medical or diagnostic procedure. As shown in FIG. 12, in some versions, the instrument lumen and channel (314) may receive a basket device (318) therein, with a basket portion (319) of basket device (318) being extendable outwardly away from distal tip member (304A) to scoop a portion of the patient's tissue or other material. The tissue or material may then be removed from the patient. The instrument lumen and channel (314) may also be used for transmitting irrigation liquid (e.g., saline, etc.) and/or medication into the patient. A user may inject an irrigation liquid into the proximal end of the instrument lumen to transmit the liquid through channel (314) and into the patient. As yet another variation, a single optics tube (302) may have several different lumens, including but not limited to a lumen for a core wire, a lumen for an instrument, a lumen for irrigation.

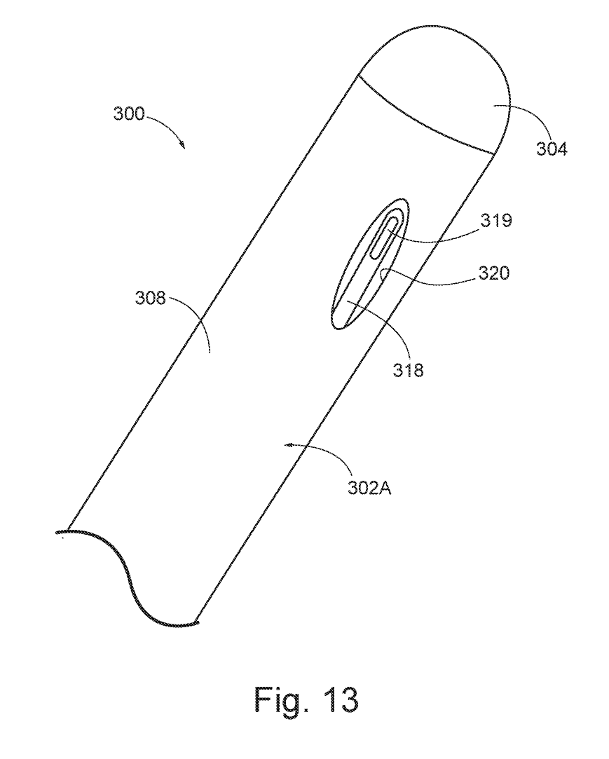

FIG. 13 shows another variation where optics tube (302) is replaced by optics tube (302A). Optics tube (302A) of this example defines a lateral opening (320) through core material (306) and cladding material (308). Opening (320) is in communication with an instrument lumen (which is offset from yet parallel with lumen (310)), whereby a tool such as basket device (318) may extend through internal lumen (310) and dispose basket portion (319) in opening (320) to collect tissue or other material from a patient. Basket device (318) may then be retracted and moved toward the proximal end of optics tube (302A) to retrieve the material.

By way of example only, optics tube (302) may have an effective outer diameter of approximately 0.020 inches to approximately 0.040 inches and an effective inner diameter of approximately 0.010 inches to approximately 0.030 inches, which may represent the approximate diameter of internal lumen (310). Optics tube (302) may have a length of approximately 10 centimeters to approximately 400 centimeters. Similarly, core wire (31) may have a length of approximately 10 centimeters to approximately 400 centimeters. Some versions of optics tube (302) may include a jacket layer (not shown) on the exterior of cladding material (308). Some versions of optics tube (302) may include a hydrophilic coating on the exterior surface of cladding material (308) or the interior surface of core material (306) defining internal lumen (310). Some other versions of optics tube (302) may include a hydrophobic coating on the exterior surface of cladding material (308) or the interior surface of core material (306) defining internal lumen (310). Of course, all of these dimensions and features are merely illustrative examples. Other suitable dimensions and features will be apparent to those of ordinary skill in the art in view of the teaching herein.

B. Capillary Light Guidewire with Multiple Lumens

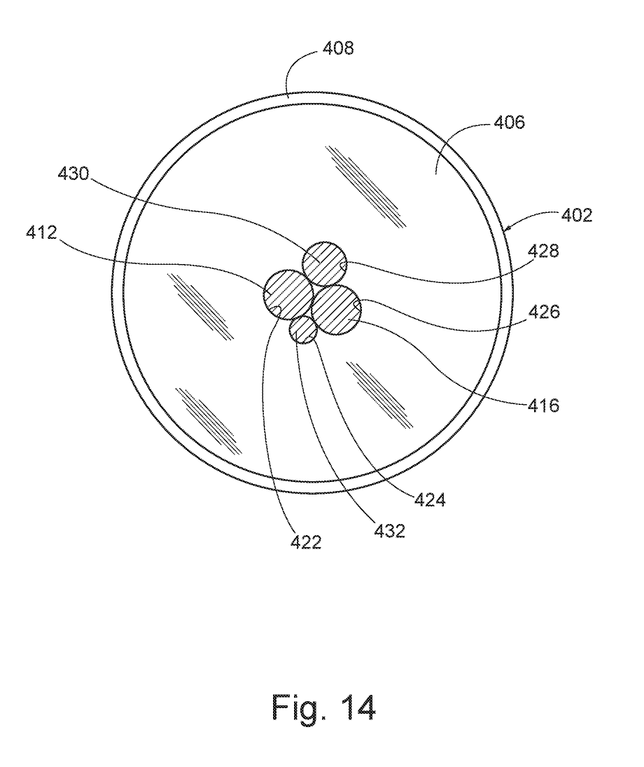

As noted above, some variations of guidewire (300) may include two or more internal lumens. FIG. 14 shows an exemplary capillary light guidewire (400) with multiple lumens that may be incorporated into dilation catheter system (100) and/or used with image guidance system (200). Except as noted herein, guidewire (400) may be constructed and operable just like guidewire (300) above. Guidewire (400) of this example comprises an optics tube (402), a core material (406), and a cladding material (408). Rather than single internal lumen (310) of guidewire (300), core material (406) of guidewire (400) defines multiple lumens within core material (406).

As shown in FIG. 14, core material (406) defines a first internal lumen (422), a second internal lumen (424), a third internal lumen (426), and a fourth internal lumen (428). While the multiple internal lumens (422, 424, 426, 428) are shown grouped generally in the central area of optics tube (402), one or more internal lumens (422, 424, 426, 428) may be disposed anywhere within core material (306). Internal lumens (422, 424, 426, 428) are configured to receive tools or devices such as a core wire (412) disposed in first internal lumen (422), a small guidewire (432) disposed in second internal lumen (424), a forceps device (416) disposed in third internal lumen (426), and an optical fiber (430) disposed in fourth internal lumen (428). Core wire (412) and forceps device (416) may be constructed and operable just like core wire (312) and forceps device (416) above, respectively. Optical fiber (430) is operable to receive images from distal tip member (not shown) of guidewire (400) and transmit this the images to an image processing device (not shown) connected to the proximal end thereof. Small guidewire (432) may be extended outwardly through distal tip member (not shown) to facilitate access to a relatively narrow anatomical passageway. In some versions, at least one of the aforementioned tools may be removed from internal lumens (422, 424, 426, 428) as desired to allow for irrigation liquids to be transmitted through an empty internal lumen (422, 424, 426, 428) into the patient.

C. Capillary Light Guidewire with Navigation Coil

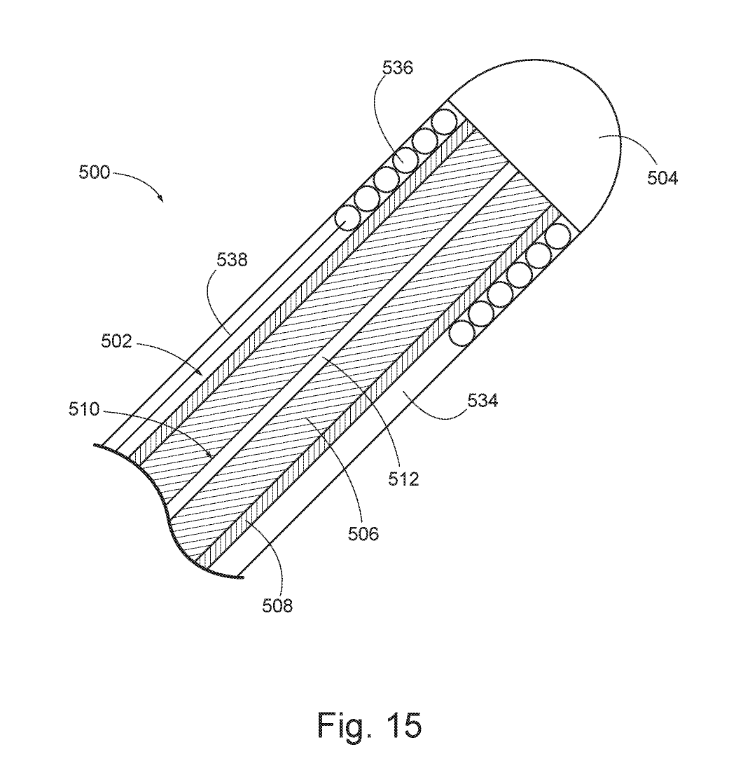

FIG. 15 shows an exemplary capillary light guidewire (500) with a navigation sensor (536) that may be incorporated into dilation catheter system (100) and/or used with image guidance system (200). Except as noted herein, guidewire (500) may be constructed and operable just like guidewires (300, 400) above. Guidewire (500) of this example comprises an optics tube (502), a distal tip member (504), an optically transmissive core material (506), a cladding material (508), a core wire (512) disposed in an internal lumen (510), and a navigation sensor (536).

Except as noted herein, navigation sensor (536) may be constructed and operable just like the navigation coil of guidewire (150). In particular, navigation sensor (536) of the present example is formed as a single-axis coil, as described in at least one of the various references cited herein. Various suitable forms that navigation sensor (536) may take will be apparent to those of ordinary skill in the art in view of the teachings herein. In the present example, navigation sensor (536) is positioned within the distal end of outer layer (534), such that navigation sensor (536) is completely exterior to optics tube (502). In some other versions, all or a portion of navigation sensor (536) may be positioned within distal tip member (504). Also in some versions, a core of iron and/or some other ferromagnetic material is positioned within the inner diameter that is defined by navigation sensor (536). Such a core of material may extend along the full length of navigation sensor (536) or a portion of the length of navigation sensor (536).

Navigation sensor (536) is configured to cooperate with image guidance system (200) to provide signals indicative of the positioning of the distal end of guidewire (500) within the patient, as described above. A navigation cable (538) is coupled with the proximal end of navigation sensor (536) and transmits the signals from navigation sensor (536) to image guidance system (200) via cable (538). It should therefore be understood that the proximal end of guidewire (500) may include a connector hub similar to connector hub (152); and that navigation cable (538) may be in communication with the connector hub.

VI. Exemplary Combinations

The following examples relate to various non-exhaustive ways in which the teachings herein may be combined or applied. It should be understood that the following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### EXAMPLE 1

An apparatus comprising: (a) an optics tube extending a proximal end to a distal end, the optics tube comprising: (i) a cladding material, (ii) a core material encased by the cladding material and configured to transmit light from the proximal end of optics tube to the distal end, and (iii) a lumen defined by the core material; (b) a distal tip member positioned proximate to the distal end of the optics tube; and (c) a core wire disposed in the lumen, wherein the core wire is configured to prevent longitudinal stretching of the optics tube.

### EXAMPLE 2

The apparatus of Example 1, wherein the core material has a first refractive index, wherein the cladding material has a second refractive index, and wherein the second refractive index is lower than the first refractive index.

### EXAMPLE 3

The apparatus of any one or more of Examples 1 through 2, wherein the cladding material is configured to reflect light.

### EXAMPLE 4

The apparatus of any one or more of Examples 1 through 3, wherein the optics tube is configured to transmit light from the proximal end to the distal end via internal reflection.

### EXAMPLE 5

The apparatus of any one or more of Examples 1 through 4, wherein the core wire is further configured to prevent kinking of the optics tube.

### EXAMPLE 6

The apparatus of any one or more of Examples 1 through 5, wherein the distal end of the core wire is secured to the distal tip member.

### EXAMPLE 7

The apparatus of any one or more of Examples 1 through 6, wherein the core wire is removably disposed in the lumen.

### EXAMPLE 8

The apparatus of any one or more of Examples 1 through 7, wherein the core wire is formed from a shape memory alloy.

### EXAMPLE 9

The apparatus of any one or more of Examples 1 through 8, further comprising an optical fiber disposed in the lumen and configured to capture images through the distal tip member.

### EXAMPLE 10

The apparatus of any one or more of Examples 1 through 9, further comprising: (a) a channel defined by the distal tip member; and (b) a forceps device configured to traverse the channel, wherein the forceps device includes a jaw, wherein the jaw is selectively extendable through the distal tip member via the channel.

EXAMPLE 11

The apparatus of any one or more of Examples 1 through 10, further comprising: (a) a channel defined by the distal tip member; and (b) a basket instrument configured to traverse the channel, wherein the basket instrument includes a basket, wherein the basket is selectively extendable through the distal tip member via the channel.

EXAMPLE 12

The apparatus of any one or more of Examples 1 through 11, further comprising a navigation sensor, wherein the navigation sensor is positioned proximate the distal end of the optics tube, wherein the navigation sensor is configured to generate a signal in response to movement of the navigation sensor within an electromagnetic field.

EXAMPLE 13

The apparatus of Example 12, further comprising an electrical wire coupled with the navigation sensor, wherein the electrical wire extends along the optics tube.

### EXAMPLE 14

The apparatus of any one or more of Examples 13 through 13, further comprising a navigation system, wherein the navigation system is operable to generate an electromagnetic field, wherein the navigation sensor is configured to generate a signal in response to movement of the navigation sensor within the electromagnetic field generated by the navigation system.

EXAMPLE 15

The apparatus of any one or more of Examples 1 through 14, wherein the lumen comprises a first lumen, the optics tube further comprising a second lumen defined by the core material.

EXAMPLE 16

An apparatus comprising: (a) an optics tube extending from a proximal end to a distal end and formed from a core material encased in a cladding material; (b) a lumen defined by the core material and extending from the proximal end to the distal end, wherein the lumen is configured to selectively receive one or more of a core wire, an optical fiber, a forceps device, or a basket device; (c) a distal tip member secured to the distal end of the optics tube; and (d) a light source operably connected to the proximal end of the optics tube, wherein the core material is configured to transmit light from the light source to the distal tip member.

EXAMPLE 17

The apparatus of Example 15, further comprising a navigation coil, wherein the navigation coil is located proximate the distal end of the optics tube, wherein the navigation coil is configured to generate a signal in response to movement of the navigation coil within an electromagnetic field.

EXAMPLE 18

The apparatus of any one or more of Examples 16 through 17, wherein the optics tube includes a hydrophilic coating on one or both of an outer surface or an inner surface wherein the inner surface defines the lumen.

EXAMPLE 19

A method comprising: (a) connecting a light source with a proximal end of an optics tube of a guidewire; (b) providing a path for communication of light from the light source through a core material of the optics tube from the proximal end to a distal end; and (c) inserting an element into a lumen defined by the core material.

EXAMPLE 20

The method of Example 19, further comprising inserting a distal end of the guidewire into a nasal cavity of a patient.

VII. Miscellaneous

It should be understood that any of the examples described herein may include various other features in addition to or in lieu of those described above. By way of example only, any of the examples described herein may also include one or more of the various features disclosed in any of the various references that are incorporated by reference herein.

It should be understood that any one or more of the teachings, expressions, embodiments, examples, etc. described herein may be combined with any one or more of the other teachings, expressions, embodiments, examples, etc. that are described herein. The above-described teachings, expressions, embodiments, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Versions of the devices disclosed herein can be designed to be disposed of after a single use, or they can be designed to be used multiple times. Versions may, in either or both cases, be reconditioned for reuse after at least one use. Reconditioning may include any combination of the steps of disassembly of the device, followed by cleaning or replacement of particular pieces, and subsequent reassembly. In particular, versions of the device may be disassembled, and any number of the particular pieces or parts of the device may be selectively replaced or removed in any combination. Upon cleaning and/or replacement of particular parts, versions of the device may be reassembled for subsequent use either at a reconditioning facility, or by a surgical team immediately prior to a surgical procedure. Those skilled in the art will appreciate that reconditioning of a device may utilize a variety of techniques for disassembly, cleaning/replacement, and reassembly. Use of such techniques, and the resulting reconditioned device, are all within the scope of the present application.

By way of example only, versions described herein may be processed before surgery. First, a new or used instrument may be obtained and if necessary cleaned. The instrument may then be sterilized. In one sterilization technique, the instrument is placed in a closed and sealed container, such as a plastic or TYVEK bag. The container and instrument may then be placed in a field of radiation that can penetrate the container, such as gamma radiation, x-rays, or high-energy electrons. The radiation may kill bacteria on the instrument and in the container. The sterilized instrument may then be stored in the sterile container. The sealed container may keep the instrument sterile until it is opened in a surgical facility. A device may also be sterilized using any other technique known in the art, including but not limited to beta or gamma radiation, ethylene oxide, or steam.

Having shown and described various versions of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, versions, geometries, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

I/We claim:

1. An apparatus comprising:
(a) an optics tube extending from a proximal end to a distal end, the optics tube comprising:
(i) a cladding material,
(ii) a core material encased by the cladding material and configured to transmit light from the proximal end of optics tube to the distal end, and
(iii) a lumen defined by the core material;
(b) a distal tip member positioned proximate to the distal end of the optics tube; and
(c) a core wire disposed in the lumen, wherein the core wire is configured to prevent longitudinal stretching of the optics tube.

2. The apparatus of claim 1, wherein the core material has a first refractive index, wherein the cladding material has a second refractive index, and wherein the second refractive index is lower than the first refractive index.

3. The apparatus of claim 1, wherein the cladding material is configured to reflect light.

4. The apparatus of claim 1, wherein the optics tube is configured to transmit light from the proximal end to the distal end via internal reflection.

5. The apparatus of claim 1, wherein the core wire is further configured to prevent kinking of the optics tube.

6. The apparatus of claim 1, wherein the distal end of the core wire is secured to the distal tip member.

7. The apparatus of claim 1, wherein the core wire is removably disposed in the lumen.

8. The apparatus of claim 1, wherein the core wire is formed from a shape memory alloy.

9. The apparatus of claim 1, further comprising an optical fiber disposed in the lumen and configured to capture images through the distal tip member.

10. The apparatus of claim 1, further comprising:
(a) a channel defined by the distal tip member; and
(b) a forceps device configured to traverse the channel, wherein the forceps device includes a jaw, wherein the jaw is selectively extendable through the distal tip member via the channel.

11. The apparatus of claim 1, further comprising:
(a) a channel defined by the distal tip member; and
(b) a basket instrument configured to traverse the channel, wherein the basket instrument includes a basket, wherein the basket is selectively extendable through the distal tip member via the channel.

12. The apparatus of claim 1, further comprising a navigation sensor, wherein the navigation sensor is positioned proximate the distal end of the optics tube, wherein the navigation sensor is configured to generate a signal in response to movement of the navigation sensor within an electromagnetic field.

13. The apparatus of claim 12, further comprising an electrical wire coupled with the navigation sensor, wherein the electrical wire extends along the optics tube.

14. The apparatus of claim 12, further comprising a navigation system, wherein the navigation system is operable to generate an electromagnetic field, wherein the navigation sensor is configured to generate a signal in response to movement of the navigation sensor within the electromagnetic field generated by the navigation system.

15. The apparatus of claim 1, wherein the lumen comprises a first lumen, the optics tube further comprising a second lumen defined by the core material.

16. An apparatus comprising:
(a) an optics tube extending from a proximal end to a distal end and formed from a core material encased in a cladding material;
(b) a lumen defined by the core material and extending from the proximal end to the distal end, wherein the lumen is configured to selectively receive one or more of a core wire, an optical fiber, a forceps device, or a basket device;
(c) a distal tip member secured to the distal end of the optics tube; and
(d) a light source operably connected to the proximal end of the optics tube, wherein the core material is configured to transmit light from the light source to the distal tip member.

17. The apparatus of claim 16, further comprising a navigation coil, wherein the navigation coil is located proximate the distal end of the optics tube, wherein the navigation coil is configured to generate a signal in response to movement of the navigation coil within an electromagnetic field.

18. The apparatus of claim 16, wherein the optics tube includes a hydrophilic coating on one or both of an outer surface or an inner surface, wherein the inner surface defines the lumen.

19. A method comprising:
(a) connecting a light source with a proximal end of an optics tube of a guidewire;
(b) providing a path for communication of light from the light source through a core material of the optics tube from the proximal end to a distal end; and
(c) inserting an element into a lumen defined by the core material.

20. The method of claim 19, further comprising inserting a distal end of the guidewire into a nasal cavity of a patient.
